(19) United States
(12) Patent Application Publication
(10) Pub. No.: US 2008/0085314 A1
(43) Pub. Date: Apr. 10, 2008

(54) Solid oral formulations for combination therapy

(76) Inventor: Shalaby W. Shalaby, Anderson, SC (US)

Correspondence Address: LEIGH P. GREGORY, PO BOX 168, CLEMSON, SC, 29633-0168, US

(21) Appl. No.: 11/494,662

(22) Filed: Jul. 27, 2006

Related U.S. Application Data
(60) Provisional application No. 60/704,018, filed on Jul. 29, 2005.

(51) Int. Cl. A61K 31/19; A61K 31/34; A61K 31/44; A61K 9/28; A61K 9/36; A61P 43/00

(52) U.S. Cl. 424/480; 424/474; 514/338; 514/471; 514/568; 514/569

(57) ABSTRACT

A first, solid oral pharmaceutical composition includes an extended release acetaminophen, a non-steroidal anti-inflammatory drug, such as naproxen or ibuprofen, and a third drug capable of reducing gastric acid secretion, such as ranitidine or omeprazole. A second, solid oral pharmaceutical composition includes a non-steroidal anti-inflammatory drug and an agent for reducing gastric acid secretion.

The present application claims the benefit of prior provisional application, U.S. Ser. No. 60/704,018 filed Jul. 29, 2005.

### FIELD OF THE INVENTION

This invention relates to novel pharmaceutical compositions of matter as solid oral formulations for combination therapy comprising an extended release acetaminophen as a principle analgesic agent admixed with a second compound as a principle anti-inflammatory agent with concomitant antipyretic and analgesic properties, plus a third compound as a principle agent for reducing gastric acid secretion. This invention also relates to another novel pharmaceutical composition of matter as a solid oral formulation for combination therapy comprising an anti-inflammatory agent and second agent for reducing gastric acid secretion. Such compositions would achieve multiple pharmacological effects of orally administered bioactive compounds while helping to preserve the biophysical integrity of the cell lining of the gastrointestinal tract and patient comfort by reducing gastric acid secretion.

### BACKGROUND OF THE INVENTION

Pain is defined as a state of physical or mental lack of well-being or uneasiness that ranges from mild discomfort to acute, often unbearable agony. Pain may be generalized or localized, and is often the consequence of being injured or hurt physically or mentally or of some derangement of or lack of equilibrium in the physical or mental environment. Analgesics are agents for reducing pain or achieving insensibility to pain without loss of consciousness. Analgesics can be of the opioid type (which can be associated with physical tolerance and/or chemical dependence) or of the non-opioid, non-addictive type. The non-opioid analgesics are most relevant to the present invention and have found use in (1) management of postoperative pain; (2) treatment of cancer pain; (3) management of neuropathic pain, which can develop after injury to any level of the nervous system; (4) management of headache, which is a common symptom of acute systemic or intracranial infection, intracranial tumor, head injuries, severe hypertension, cerebral hypoxia and many diseases of the eye, nose, teeth, and ears; and (5) treatment of somatic or visceral pain in acutely or terminally ill patients.

In a recent disclosure (U.S. Pat. No. 6,492,334) it was noted that (1) until 1955, aspirin was the primary over-the-counter drug used for treating pain, but long-term use of this drug may lead to bleeding and ulceration; (2) acetaminophen was later introduced as an analgesic without the side effects of aspirin; and (3) acetaminophen has no effect on inflammation and it can only be used as an analgesic. In spite of the latter statement, acetaminophen has been described as a weak anti-inflammatory drug (AHFS Drug Information, American Society of Health System Pharmacists, Bethesda, MD, 1999, p. 1826) for some non-rheumatoidal conditions (oral surgery). Nevertheless, for treating pain associated with inflammation, formulations containing acetaminophen and a highly effective anti-inflammatory drug are warranted. The present invention addresses the need, in part, to provide a combination of an anti-inflammatory drug and acetaminophen with an extended release profile that exceeds those of the commercial formulations where a mere enteric coating of the tablets is used to control the drug release. Towards exploring a new combination therapy formulation that would include a non-steroidal, anti-inflammatory drug (NSAID), two commonly used and highly effective NSAIDs, ibuprofen and naproxen, were selected for use in the compositions subject of the present invention. Both naproxen and ibuprofen are believed to exhibit their established anti-inflammatory effect through inhibiting the synthesis of prostaglandin. In addition to their strong anti-inflammatory effect, ibuprofen and naproxen display, to a lesser extent, antipyretic and analgesic properties which supplement those noted for acetaminophen. More specifically, a combination drug product having ibuprofen will be useful in treating a variety of painful maladies in addition to inflammatory diseases including, but not limited to, acute pain, osteoarthritis and rheumatoid arthritis. A similar combination of acetaminophen and naproxen will be effective in treating the aforementioned disease states. Unfortunately, both ibuprofen and naproxen, which contain carboxylic or carboxylate groups, can cause gastric mucosal abnormalities including edema and erythema. And it has been noted in the prior art (U.S. Pat. No. 6,710,086) that there exist two major ulcerogenic effects of the NSAIDs (including ibuprofen and naproxen) namely, (1) topical irritant effect on the epithelium of the gastrointestinal tract and (2) suppression of gastrointestinal prostaglandin synthesis. It was also noted that attempts to reduce the damage to the gastrointestinal tract through using tablets with enteric coating or developing slow-release formulations to reduce the topical irritant properties of NSAIDs have been largely unsuccessful. This created a definite need to develop an analgesic/anti-inflammatory pharmaceutical formulation comprising an agent that is capable of (1) reducing the gastric acid secretion and (2) counteracting the ulcerogenic effect of the NSAID in such a formulation.

### SUMMARY OF THE INVENTION

Accordingly, this invention deals with a multifunctional formulation for concomitant oral administration of (1) acetaminophen as an extended release, strong analgesic; (2) ibuprofen or naproxen as a strong anti-inflammatory/analgesic agent; and (3) an agent, such as omeprazole or ranitidine, for reducing the gastric acid secretion to help exert an anti-ulcerative effect on the mucosal surface. More specifically, omeprazole (1) is a strong gastric anti-secretory agent commonly referred to as a proton pump inhibitor; (2) inactivates the adenosine triphosphatase enzyme system in gastric parietal cells and blocks the final step in the secretion of hydrochloric acid by these cells; and (3) it inhibits basal and stimulated gastric acid secretions. On the other hand, ranitidine (1) is a histamine H₂-receptor antagonist; (2) competitively inhibits the action of histamine on the H₂-receptor of parietal cells, reducing gastric acid secretion under daytime and nocturnal basal conditions and also when stimulated by food; and (3) is effective in treating duodenal and gastric ulcers as well as gastroesophageal reflux. This invention also deals with unexpected retention of the chemical integrity of the individual drugs present in 3-drug formulations and absence of any interaction by-products commonly encountered in structurally unrelated combinations of functional chemical compounds.

Acknowledging that incorporating a gastric acid-reducing agent with an anti-inflammatory drug for general use in combination therapy, as discussed above, provided the incentive to pursue a second scheme for a new, drug formulation. Accordingly, this invention also deals with a solid pharmaceutical formulation for relieving pain without contending with gastric acid-related complications and consisting of a non-steroidal anti-inflammatory drug and an agent for reducing gastric acid secretion.

Accordingly, the present invention is directed to a solid oral pharmaceutical composition comprising (a) a controlled release acetaminophen, (b) an NSAID, and (c) an agent capable of reducing gastric acid secretion. The NSAID is selected from the group represented by naproxen, naproxen sodium, ketoprofen, ibuprofen, and ibuprofen sodium, and the agent capable of reducing gastric acid secretion is selected from the group represented by ranitidine hydrochloride, ranitidine immobilized on cation-exchanging absorbable microparticles, famotidine, cimetidine, and omeprazole.

Another aspect of this invention relates to a solid oral pharmaceutical composition comprising (a) a controlled release acetaminophen, (b) an NSAID, and (c) an agent capable of reducing gastric acid secretion, wherein the composition is in the form of a coated tablet comprising coated granules of acetaminophen and uncoated microparticles of a non-steroidal anti-inflammatory and an agent for reducing the gastric acid secretion, wherein the tablet has an enteric coating comprising a film-forming cellulose derivative.

A specific aspect of this invention deals with a solid oral pharmaceutical composition comprising (a) a controlled release acetaminophen, (b) an NSAID, and (c) an agent capable of reducing gastric acid secretion, wherein the composition is contained in a capsule wherein the acetaminophen is part of an absorbable fibrous composite, the agent for reducing gastric acid secretion is in the form of uncoated microparticles and the non-steroidal anti-inflammatory drug is in the form of uncoated granules.

Another aspect of this invention deals with a solid oral pharmaceutical composition comprising (a) a controlled release acetaminophen, (b) an NSAID, and (c) an agent capable of reducing gastric acid secretion, wherein the composition is contained in a capsule wherein the acetaminophen is part of composite film comprising a cellulose derivative, the agent for reducing gastric acid secretion is in the form of uncoated microparticles and non-steroidal anti-inflammatory drug is in the form of uncoated granules.

A general aspect of this invention deals with a method for the treatment of pain, inflammation, injury, arthritis, trauma, and fever while protecting the cell lining of the digestive tract in a person in need of such treatment comprising administering to said person an effective amount of (a) acetaminophen, (b) a non-steroidal anti-inflammatory drug, and (c) an agent for reducing gastric acid secretion.

An important aspect of this invention deals with a solid oral pharmaceutical composition comprising (a) a controlled release acetaminophen, (b) a non-steroidal anti-inflammatory drug, and (c) an agent capable of reducing gastric acid secretion, wherein said composition is in the form of a bilayered tablet comprising two compressed discs, one containing acetaminophen while the other comprising a non-steroidal anti-inflammatory drug and an agent capable of reducing gastric acid secretion. More specifically, the non-steroidal anti-inflammatory drug is selected from the group represented by naproxen, naproxen sodium, ibuprofen, and ibuprofen sodium and the agent for reducing gastric acid secretions is selected from the group represented by ranitidine hydrochloride, ranitidine immobilized on absorbable cation-exchanging microparticles, and omeprazole.

Another aspect of this second invention deals with a solid oral pharmaceutical composition comprising (a) a controlled release acetaminophen, (b) a non-steroidal anti-inflammatory drug, and (c) an agent capable of reducing gastric acid secretion, wherein said composition may be the form of a bilayered tablet comprising two adjoined compressed discs, one containing acetaminophen while the other comprising a non-steroidal anti-inflammatory drug and an agent capable of reducing gastric acid secretion, and wherein the bilayered tablet is made according to the following steps: (a) acetaminophen is mixed with a mixture of low-melting ethylene-vinyl acetate copolymer, a solid high molecular weight polyethylene glycol, and optional excipients; (b) acetaminophen formulation is then heat-pressed into the first disc; and (c) the acetaminophen-containing disc is heat-pressed onto a mixture of the non-steroidal anti-inflammatory drug, the agent for reducing gastric acid secretion and optional excipients to form the adjoining discs of the bilayered tablet. Additionally, the bilayered tablet may have an enteric coating comprising a film-forming cellulose derivative.

A major segment of this invention deals with a solid oral pharmaceutical composition comprising (a) a non-steroidal anti-inflammatory drug; (b) an agent capable of reducing gastric acid secretion; and (c) optional excipients, wherein the non-steroidal anti-inflammatory drug is selected from the group represented by naproxen, naproxen sodium, ibuprofen and ibuprofen sodium, and wherein the agent for reducing gastric acid secretion is selected from the group represented by ranitidine hydrochloride, ranitidine immobilized on absorbable cation-exchanging microparticles, and omeprazole. Additionally, this composition may be in the form of a bilayered tablet comprising two adjoined compressed discs, one containing the non-steroidal anti-inflammatory drug and at least one excipient while the other comprising the agent for reducing gastric acid secretion and at least one excipient, wherein the non-steroidal anti-inflammatory drug is selected from the group represented by naproxen, naproxen sodium, ibuprofen, and ibuprofen sodium, and wherein the agent for reducing gastric acid secretion is selected from the group represented by ranitidine hydrochloride, ranitidine immobilized on absorbable cation-exchanging microparticles, and omeprazole.

From a processing perspective, the bilayered tablet is made according to the following steps: (a) each drug is heat-pressed into a disc comprising one of the drugs, a mixture of a solid polyethylene glycol, and an ethylene-vinyl acetate copolymer and (b) two discs are adjoined by heat-pressing, wherein the bilayered tablet may have an enteric coating comprising a film-forming cellulose derivative.

A clinically relevant aspect of this invention deals with a method for the treatment of inflammation, injury, arthritis, trauma, and fever while protecting the cell lining of the digestive tract in a person in need of such treatment comprising administering to said person an effective amount of (a) a non-steroidal anti-inflammatory drug; and (b) an agent for reducing gastric acid secretion.

### DETAILED DESCRIPTION OF PREFERRED EMBODIMENTS

Of the available over-the-counter drugs used primarily as an analgesic for relieving pain, acetaminophen has proven to be safe and effective, particularly when formulated to exhibit extended release. One major aspect of this invention deals with a new composition of acetaminophen that is formulated to prolong the drug release profile beyond those associated with commercially available products and hence, extend the bioavailability of the subject analgesic. For patients who suffer from different forms of inflammation, a strong NSAID, such as naproxen or ibuprofen, are known to be most effective in treating a variety of inflammation states arising from trauma, arthritis, cancer, surgery, and the different types of diseases as outlined in the Background of this invention. An important aspect of this invention deals with the novel use of acetaminophen in combination with an over-the-counter NSAID, such as naproxen and ibuprofen (as the free acid and preferably the sodium salt) to achieve combination therapy (or double therapy) for treating pain and inflammation. Recognizing the problems of carboxyl-bearing orally administered drugs in compromising the parietal cells of the gastrointestinal tract, which can lead to ulceration, this invention is directed to adding a third bioactive agent to the analgesic/NSAID combination that is capable of reducing the gastric acid secretion and hence, achieving a triple therapy. Of the agents capable of reducing gastric acid secretion, typical over-the-counter drugs are selected for use in the composition subject of this invention. These include ranitidine hydrochloride as well as other salts or complexes thereof, and omeprazole and complexes or salts thereof.

More specifically, one major segment of this invention deals with a solid oral pharmaceutical composition A which is a combination of (a) a controlled release acetaminophen, (b) an NSAID, and (c) an agent capable of reducing gastric acid secretion, wherein the NSAID is selected from the group represented by naproxen, naproxen sodium, ketoprofen, ibuprofen, and ibuprofen sodium. The agent capable of reducing gastric acid secretion is selected from the group represented by ranitidine hydrochloride, ranitidine immobilized on cation-exchanging absorbable microparticles, famotidine, cimetidine, and omeprazole. A second embodiment of this invention deals with a two-drug solid pharmaceutical composition B which is a combination of an NSAID and an agent for reducing gastric acid secretion. The NSAID is selected from the group represented by naproxen, naproxen sodium, ketoprofen, ibuprofen, and ibuprofen sodium. The agent for reducing gastric acid secretion is selected from the group represented by ranitidine hydrochloride, ranitidine immobilized on absorbable cation-exchanging microparticles (or powder), famotidine, cimetidine, and omeprazole and complexes or salts thereof. Each of these compositions A and B is denoted as Theraprene™ by Poly-Med, Inc., Anderson, S.C.

Given below is a brief discussion to further illustrate the scientific, technological, clinical, and commercial significance of the subject of this invention as it relates to combination therapy for the treatment of pain.

Pain, both acute and chronic, is one of the most common reasons for patients to visit their physician or pharmacist. There are a host of agents available, both prescription and over-the-counter, that alleviate pain and/or inflammation. As with all medications, there are advantages and disadvantages to each medication class. As such, the benefits of taking a particular medication must be weighed against the risks.

Narcotic agonists have a long history of use as analgesics. And while they are extremely effective at alleviating pain, their side effect profile and their potential for tolerance and addiction limits their use as first-line therapy. Acetaminophen has long been recognized as a safe analgesic agent for minor pain. Worldwide, it is one of the most commonly used medications for pain. And while acetaminophen in therapeutic doses is extremely well tolerated and safe as an analgesic, it has minimal effect on the inflammatory process.

Non-steroidal anti-inflammatory agents (NSAIDs) are also one of the most common medications taken for pain. It has been estimated that in the United States, one in seven Americans is likely to be treated with an NSAID for a chronic rheumatologic disorder. In fact, more than 1% of the population takes these drugs on a daily basis. Additionally, there are more than 80 million prescriptions are written for NSAIDs every year in the United States, which accounts for 4.5% of all prescriptions written. Worldwide, more than 30 million people consume NSAIDs daily.

NSAIDs are potent analgesics that have the added benefit of being able to reduce the signs and symptoms of inflammation. NSAIDs act by competitive inhibition of the Cyclooxygenase (COX) enzyme, thereby blocking prostaglandin synthesis. The COX enzyme exists in two isoforms, COX-1 and COX-2. The COX-1 isoenzyme is constitutively expressed in virtually all tissues and is responsible for the production of “maintenance prostaglandins” that function to maintain normal cell function. Such maintenance functions include vascular hemostasis, autocrine response to circulating hormones, and the production of a protective layer over the gastric mucosa to protect it from the harsh acidic environment. On the other hand, the COX-2 isoform exists mainly as an inducible enzyme that is upregulated by migratory cells and damaged tissues by pro-inflammatory stimuli. The COX-2 isoform then serves to enhance the inflammatory process. Inhibition of the COX-2 enzyme is considered the therapeutic effect of NSAIDs, while inhibition of COX-1 is considered a side effect. Unfortunately, traditional NSAIDs are not selective in their inhibition of the COX isoenzymes, and as a result, efforts to minimize inflammation through COX-2 inhibition are done at the expense of COX-1 inhibition. NSAID-induced COX-1 inhibition can lead to gastritis or peptic ulcer formation making NSAIDs a common cause of gastrointestinal bleeding. What is more concerning, however, is that many of these bleeding events occur without any warning or preceding symptoms.

In order to subvert the gastrointestinal side effects of traditional NSAIDs, much work has been done to develop agents that selectively block COX-2 while sparing COX-1. Such agents, known as COX-2 inhibitors, have been marketed as safe and effective anti-inflammatories. These agents were shown to be as effective as traditional NSAIDs in controlling pain and inflammation with less gastrointestinal toxicity. These medications became extremely popular and were beginning to supplant traditional NSAIDs. Unfortunately, recent studies have shown that COX-2 inhibitors may be linked to increased cardiovascular morbidity and mortality. As such, two of the three marketed COX-2 inhibitors in the United States were voluntarily withdrawn from the market.

In accordance with the present invention, the combination of sustained release acetaminophen and naproxen sodium allows for convenient dosing schedules, provide analgesia via two separate mechanisms, and imparts an anti-inflammatory effect as well. As naproxen sodium is a traditional NSAID, it too carries the risk of gastrointestinal toxicity by nonselective inhibition of the COX isoenzymes. To counteract the potential for side effects, the present preferred formulation also contains ranitidine. This agent acts by inhibiting the H2 receptor and leads to decreased acid production in the stomach. “H2-blockers,” such as ranitidine, have long been used in the treatment of gastritis, peptic ulcer disease, and esophagitis by helping temper acid production in the stomach, thus limiting the potential for mucosal damage. Co-administration of an H2 blocker would lead to a decrease in acid production and allow for effective doses of naproxen sodium to be delivered with less potential for mucosal damage. Several recent studies have evaluated the benefits of co-administering prophylactic acid-suppressing medications with NSAIDs. These studies have shown significantly less gastrointestinal toxicity and bleeding events.

Combination therapy like that seen in Theraprene can lead to enhanced analgesia and anti-inflammatory effects with less risk gastrointestinal toxicities. Given that often patients who have gastrointestinal bleeds from NSAIDS have no preceding symptoms, it is vital that these agents be co-administered with a prophylactic acid-suppressing agent such as ranitidine. The combination formulation can provide analgesia, anti-inflammatory effects with a much safer safety profile than any single agent. Combining these agents into a single pill obviates the need to take an addition pill for prophylactic acid suppression. This is an important advantage in the treatment of both acute and chronic pain.

A general aspect of this invention deals with a method for the treatment of pain, inflammation, injury, arthritis, trauma, and fever while protecting the cell lining of the digestive tract in a person in need of such treatment comprising administering to said person an effective amount of a solid oral pharmaceutical composition which is a combination of (a) acetaminophen, (b) a non-steroidal anti-inflammatory drug, and (c) an agent for reducing gastric acid secretion or administering a solid oral pharmaceutical composition which is a combination of a non-steroidal anti-inflammatory drug and an agent for reducing gastric acid secretion.

Further illustrations of the present invention are provided by the following examples:

### EXAMPLE 1

### Preparation of an Acetaminophen Extended Release System Using a Polyether-ester as a Matrix

The polyether ester used as a matrix for acetaminophen is made by end-grafting ε-caprolactone (70 g) on polyethylene glycol having a molecular weight of 35 kDa (30 g) in the presence of stannous octanoate as a catalyst (monomer and catalyst ratio=2,000) at 160° C. until complete conversion is achieved as determined by GPC. Part of the resulting polymer (5 g) was dissolved in a solution of acetaminophen (5 g) in acetone (100 mL). The solution was cast on a release paper and allowed to dry first at 25° C. and then reduced pressure until a constant weight is realized.

### EXAMPLE 2

### Preparation of an Acetaminophen Extended Release System Using Hydroxypropyl Methyl Cellulose Phthalate as a Matrix

The composite film based on 50% acetaminophen in high molecular weight hydroxypropyl methyl cellulose phthalate is made under conditions similar to those described in Example 1, with the exception of using a 1:1 mixture of acetone and methanol to dissolve the polymer and acetaminophen.

### EXAMPLE 3

### Characterization of a Solution of Acetaminophen with Naproxen Sodium and Ranitidine Hydrochloride

To verify the absence of any new species formed due to any possible chemical interaction of the three drugs, the following analytical protocol was pursued. Equal amounts of acetaminophen, naproxen sodium, and ranitidine hydrochloride were dissolved in methanol to produce a solution containing 0.05 mg/mL of each of the drugs. The mixture was analyzed by reverse phase HPCL using a C-18 column and a gradient immobile phase of acetonitrile and water containing 0.1% trifluoroacetic acid. Using standard curves prepared earlier for each of the drugs following identical conditions to those used in analyzing the mixture indicated that the concentrations of the individual drugs in this mixture are the same as expected in the absence of any chemical interaction.

### EXAMPLE 4

### Characterization of a Solution of Acetaminophen, Ibuprofen, and Ranitidine Hydrochloride

Analytical verification of the absence of any new species formed due to any chemical interaction of the three drugs was conducted as described in Example 3. And results showed each of the drugs retained its original chemical identity in the dissolved mixture.

### EXAMPLE 5

### Characterization of a Solution of Acetaminophen, Naproxen, and Omeprazole

Analytical verification of the absence of any new species formed due to any chemical interaction of the three drugs was conducted as described in Example 3. And results showed each of the drugs retained its original chemical identity in the dissolved mixture.

### EXAMPLE 6

### Characterization of a Solution of Acetaminophen, Ibuprofen, and Omeprazole

Analytical verification of the absence of any new species formed due to interaction of the three drugs was conducted as described in Example 3. And results showed each of the drugs retained its original chemical identity in the dissolved mixture.

### EXAMPLE 7

### Preparation of a Capsule for the Oral Administration of Extended Release of Acetaminophen, Naproxen Sodium, and Ranitidine Hydrochloride

The extended release composite film of Example 1 (650 mg) is inserted in the male compartment of a gelatin capsule. To this was added naproxen sodium granules (110 mg) and ranitidine hydrochloride microparticles (37.5 mg). The female component of the gelatin capsule is then assembled with the male component for use in the study of the release profile of the three drugs.

### EXAMPLE 8

### Preparation of a Capsule for the Oral Administration of an Extended Release of Acetaminophen, Naproxen Sodium, and Omeprazole

The extended release composite film of Example 1 (650 mg) is inserted in the male compartment of a gelatin capsule. To this was added naproxen sodium granules (110 mg) and omeprazole microparticles (5 mg). The female component of the gelatin capsule is then assembled with the male component for use in the study of the release profile of the three drugs.

### EXAMPLE 9

### Preparation of a Capsule for Oral Administration of an Extended Release Acetaminophen, Ibuprofen, and Ranitidine Hydrochloride

The extended release composite film of Example 1 (650 mg) is inserted in the male compartment of a gelatin capsule. To this was added ibuprofen (25 mg) and ranitidine hydrochloride microparticles (37.5 mg). The female component of the gelatin capsule is then assembled with the male component for use in the study of the release profile of the three drugs.

### EXAMPLE 10

### Preparation of a Capsule for Oral Administration of Extended Release Acetaminophen, Ibuprofen, and Omeprazole

The extended release composite film of Example 1 (650 mg) is inserted in the male compartment of a gelatin capsule. To this was added ibuprofen (25 mg) and omeprazole (3.33 mg). The female component of the gelatin capsule is then assembled with the male component for use in the study of the release profile of the three drugs.

### EXAMPLE 11

### Preparation and Study of a Three-drug Bilayered Tablet Formulation I

To prepare a bilayered tablet of acetaminophen naproxen sodium, and ranitidine, HCl, the following steps are pursued. Eight grams of solid polyethylene glycol (PEG 4600) and twelve grams of acetaminophen are placed in a flask and heated to about 80° C. to melt and mix. Once the mixture has cooled, 4 grams of ethylene-vinyl acetate copolymer (Elvax 210) is added and mechanically stirred at about 90° C. The resulting mixture comprises the acetaminophen base portion of the tablet. The acetaminophen base (650 g) is heat-pressed in a special ½ inch diameter mold at about 60° C. Onto this is added a thoroughly ground mixture of 38 mg of ranitidine HCl, 110 mg of naproxen sodium, and 130 mg of PEG 2000, and is heat-pressed at about 70° C. to form the bilayered tablet.

To study the release profile of the three drugs, the tablet is placed in simulated gastric fluid for the first hour and then deionized for an additional 7 hours. The release profile is determined using HPLC analysis of the aqueous medium and indicates that the ranitidine HCl and naproxen sodium are released within the first two hours and the acetaminophen displays a controlled release profile over an 8-hour period.

### EXAMPLE 12

### Preparation and Study of a Three-drug bilayered Tablet Formulation II

To prepare Formulation II as a bilayered tablet comprising acetaminophen, ibuprofen sodium, and ranitidine HCl, a procedure similar to that used in Example 11 is followed with the exception of using 100 mg of ibuprofen sodium and 25 mg of ranitidine HCl.

The release profile of the resulting bilayered tablet of Formulation II is determined as described in Example 11. The results show that ranitidine HCl and ibuprofen sodium release in the first 2 hours and acetaminophen is released over an 8-hour period.

Preferred embodiments of the invention have been described using specific terms and devices. The words and terms used are for illustrative purposes only. The words and terms are words and terms of description, rather than of limitation. It is to be understood that changes and variations may be made by those of ordinary skill art without departing from the spirit or scope of the invention, which is set forth in the following claims. In addition it should be understood that aspects of the various embodiments may be interchanged in whole or in part. Therefore, the spirit and scope of the appended claims should not be limited to descriptions and examples herein.

What is claimed is:

1. A solid oral pharmaceutical composition comprising (a) a controlled release acetaminophen, (b) a non-steroidal anti-inflammatory drug, and (c) an agent capable of reducing gastric acid secretion.

2. The composition of claim 1, wherein the non-steroidal anti-inflammatory drug is selected from the group consisting of naproxen, naproxen sodium, ibuprofen, and ibuprofen sodium.

3. The composition of claim 1 wherein the agent capable of reducing gastric acid secretion is selected from the group consisting of ranitidine hydrochloride, ranitidine immobilized on absorbable cation-exchanging microparticles and omeprazole.

4. The composition according to claim 1, wherein the composition is in the form of a coated tablet comprising coated granules of acetaminophen and uncoated microparticles of a non-steroidal anti-inflammatory agent for reducing the gastric acid secretion.

5. The composition as set forth in claim 4 wherein the tablet has an enteric coating comprising a film-forming cellulose derivative.

6. The composition according to claim 1 contained in a capsule wherein the acetaminophen is part of an absorbable fibrous composite, the agent for reducing gastric acid secretion is in the form of uncoated microparticles and the non-steroidal anti-inflammatory drug is in the form of uncoated granules.

7. The composition according to claim 1 contained in a capsule wherein the acetaminophen is part of composite film comprising a cellulose derivative, the agent for reducing gastric acid secretion is in the form of uncoated microparticles and non-steroidal anti-inflammatory drug is in the form of uncoated granules.

8. The composition according to claim 1 where said composition is in the form of a bilayered tablet comprising two adjoined compressed discs, one disc containing acetaminophen, the other disc comprising a non-steroidal anti-inflammatory drug and an agent capable of reducing gastric acid secretion.

9. The composition of claim 8 wherein the non-steroidal anti-inflammatory drug is selected from the group consisting of naproxen, naproxen sodium, ibuprofen, and ibuprofen sodium.

10. The composition of claim 8 wherein the agent for reducing gastric acid secretions is selected from the group consisting of ranitidine hydrochloride, ranitidine immobilized on absorbable cation-exchanging microparticles, and omeprazole.

11. The composition of claim 8 wherein the bilayered tablet has an enteric coating comprising a film-forming cellulose derivative.

12. A method for the treatment of inflammation, injury, arthritis, trauma, and fever while protecting the cell lining of the digestive tract in a person in need of such treatment comprising administering to said person an solid oral pharmaceutical composition comprising an effective amount of (a) acetaminophen; (b) a non-steroidal anti-inflammatory drug; and (c) an agent for reducing gastric acid secretion.

13. A solid oral pharmaceutical composition comprising (a) a non-steroidal anti-inflammatory drug; (b) an agent capable of reducing gastric acid secretion; and (c) optional excipients.

14. The composition of claim 13 wherein the non-steroidal anti-inflammatory drug is selected from the group consisting of naproxen, naproxen sodium, ibuprofen, and ibuprofen sodium.

15. The composition of claim 13 wherein the agent for reducing gastric acid secretion is selected from the group consisting of ranitidine hydrochloride, ranitidine immobilized on absorbable cation-exchanging microparticles, and omeprazole.

16. A composition of claim 13 in the form of a heat-pressed tablet comprising a solid high molecular weight polyethylene glycol and an ethylene vinyl acetate copolymer as excipients.

17. The composition of claim 13 wherein the tablet has an enteric coating comprising a film-forming cellulose derivative.

18. The composition of claim 13 wherein said composition is in the form of a bilayered tablet comprising two adjoined compressed discs, one containing the non-steroidal anti-inflammatory drug and at least one excipient, the other comprising the agent for reducing gastric acid secretion and at least one excipient.

19. The composition of claim 18 wherein the non-steroidal anti-inflammatory drug is selected from the group consisting of naproxen, naproxen sodium, ibuprofen, and ibuprofen sodium.

20. The composition of claim 18 wherein the agent for reducing gastric acid secretion is selected from the group consisting of ranitidine hydrochloride, ranitidine immobilized on absorbable cation-exchanging microparticles, and omeprazole.

21. The composition as in claim 18 wherein the bilayered tablet has an enteric coating comprising a film-forming cellulose derivative.

22. A method for the treatment of inflammation, injury, arthritis, trauma, and fever while protecting the cell lining of the digestive tract in a person in need of such treatment comprising administering to said person a solid oral pharmaceutical composition comprising an effective amount of (a) a non-steroidal anti-inflammatory drug; and (b) an agent for reducing gastric acid secretion.
